(19) United States
(12) Patent Application Publication
(10) Pub. No.: US 2019/0225694 A1
(43) Pub. Date: Jul. 25, 2019

(54) RECOMBINANT PRODUCTION OF MONOCLONAL ANTIBODIES

(71) Applicant: ZAKLADY FARMACEUTYCZNE POLPHARMA SA, Starogard Gdanski (PL)

(72) Inventors: Piotr Marcin Zien, Sadowa (PL); Matthew Christopher Cheeks, London (GB); Tomasz Sitar, Gdansk (PL); Kornelia Bogumila Wisniewska, Gdansk (PL); Rafal Andrzej Derlacz, Warszawa (PL)

(21) Appl. No.: 16/313,377

(22) PCT Filed: Jun. 27, 2017
(86) PCT No.: PCT/EP2017/065831

§ 371 Date: Dec. 26, 2018

(30) Foreign Application Priority Data
Jun. 28, 2016 (EP) 16460039.7

(51) Int. Cl. C07K 16/28; C12N 5/071

(52) U.S. Cl. CPC C07K 16/2842; C12N 5/0682; C12N 2510/02; C07K 2317/24; C07K 2317/41; C07K 2317/14

(57) ABSTRACT

The present invention is directed to a cell culture obtainable from CHO DG44 cells which are capable of being cultured under serum-free or protein-free culture conditions, and which express a biosimilar antibody for the monoclonal antibody natalizumab. The present invention is further directed to a cell of said cell culture, a method for producing said biosimilar antibody, and the use of said cell in said method.

The present invention is directed to a cell culture obtainable from CHO DG44 cells which are capable of being cultured under serum-free or protein-free culture conditions, and which express a biosimilar antibody for the monoclonal antibody natalizumab. The present invention is further directed to a cell of said cell culture, a method for producing said biosimilar antibody, and the use of said cell in said method.

### BACKGROUND OF THE INVENTION

The recombinant therapeutic monoclonal antibody natalizumab is an IgG4 full-length antibody humanized from a murine monoclonal antibody that binds to the α4β1 integrin (also known as VLA-4 or CD49d-CD29) and α4β7 integrin, and blocks the interaction of said α4 integrins with their respective receptors VCAM-1 and MadCAM-1 which are expressed on endothelial cells. See also WO 95/19790. α4-integrin is required for inflammatory lymphocytes to attach to and pass through the cell layers lining the intestine and blood-brain-barrier.

Natalizumab is marketed by Biogen Idec and Elan under the name Tysabri, and was previously named Antegren. It has FDA-approval for the treatment of multiple sclerosis and Crohn's disease, and EMEA approval for the treatment of multiple sclerosis. Recently, it was suggested that natalizumab could also be used in a combination treatment of B-cell malignancies, where it is intended to overcome the resistance to rituximab. Natalizumab is typically administered by intravenous infusion. According to the Scientific Discussion available from the EMEA, natalizumab is recombinantly produced in a NS/0 murine myeloma cell line. The antibody is then purified using Protein A affinity chromatography and hydrophobic interaction chromatography, followed by a buffer exchange and concentration by ultrafiltration/diafiltration.

Currently, cell line development technologies used by most biopharmaceutical companies are based on either the methotrexate (MTX) amplification technology that originated from the 1980's, or Lonza's glutamin synthetase (GS) system. Both systems make use of a specific drug to inhibit a selectable enzyme marker essential for cellular metabolism: MTX inhibits dihydrofolate reductase (DHFR) in the MTX amplification system, and methionine sulphoximine (MSX) inhibits GS in the GS system. Upon optimisation of culture conditions, values of 2.7 g/l of monoclonal antibody have recently been reported for GS-NS0 cells in fedbatch culture (Zhou et al., Biotechnology and Bioengineering, 55(5): 783-792 (1997)). Methods for high density cell cultures of NS0 cells for, inter alia, producing natalizumab is disclosed in WO 2013/006461.

While it is generally desirable to increase product titers, therapeutic monoclonal antibodies (mAbs) that are produced in specific cell line expression systems possess inherent post-translational modification profiles which are characteristic of that host cell line. In particular N-linked glycosylation profiles can vary greatly based on the cell line expression system. Glycosylation patterns dictate the stability and functionality of the resulting glycoconjugates. Glycosylation confers functional diversity to a protein, and defective glycosylation of proteins often leads to inactive or abnormal proteins that may result in defects in cellular processes, including those in development, immune reactions, and cell signaling pathways.

The goal of biocomparability/bioequivalence (BC/BE) testing is to demonstrate that variation between different formulations or manufacturing processes do not affect the “quality, safety and efficacy of the drug product” during development or post-marketing (FDA, 2005).

Since the original CHO cell line was described in 1956, many variants of the cell line have been developed. In one strain, CHO DG44, both alleles of the DHFR locus were completely eliminated (Urlaub et al. Cell, 33: 405-412 (1983)). However, it was characterized only as being DHFR deficient and was not named (“eleven of 12 clones screened”, page 408). DG44 has been first named and characterized in Urlaub et al., Somatic Cell and Molec. Genet., 12: 555-566 (1986). These DHFR-deficient strains require glycine, hypoxanthine, and thymidine for growth.

WO 2009/009523 is directed to the prevention of disulphide bond reduction during recombinant production of polypeptides. A preferred host cell is CHO cell line DP12. Anti-human α4β7 is mentioned in a washing list of antibodies, which may be produced using the disclosed method.

WO 2011/019619 discloses the use of DHFR⁻ CHO host cell lines in the production of monoclonal antibodies.

EP 2 202 307 A1 describes the production of antibodies using, inter alia, CHO cells harbouring an enlarged number of copies of the DNA encoding said antibody.

The object of the invention was to provide a host cell expression system for a biosimilar of natalizumab. In particular, the object was to provide an expression system providing higher production yields while maintaining quality, safety and efficacy of the drug product.

### SUMMARY OF THE INVENTION

The present inventors have surprisingly found that CHO DG44 cells can be used for producing a monoclonal antibody which is a biosimilar to the therapeutic antibody natalizumab. In particular, it was surprisingly found that similar glycosylation patterns can be obtained when using this CHO cell strain. Glycosylation plays a predominant role in determining the function, pharmacokinetics, pharmacodynamics, stability, and immunogenicity of biotherapeutics. There are many physical functions of N-linked glycosylation in a mAb such as affecting its solubility and stability, protease resistance, binding to Fc receptors, cellular transport and circulatory half-life in vivo. At the same time, the production strain shows a high a peak viable cell concentration and achieves a productivity which is higher than the productivity reported for NS0 expression systems.

Accordingly, the present invention provides a cell culture obtainable from CHO DG44 cells which are capable of being cultured under serum-free or protein-free culture conditions, and which express a polypeptide comprising amino acids 19 to 231 of SEQ ID NO: 2 and a polypeptide comprising amino acids 19 to 468 of SEQ ID NO: 4. In a preferred embodiment, the cells express a polypeptide comprising the amino acid sequence of SEQ ID NO: 2 and a polypeptide comprising the amino acid sequence of SEQ ID NO: 4; more preferably the cells express a polypeptide consisting of the amino acid sequence of SEQ ID NO: 2 and a polypeptide consisting of SEQ ID NO: 4.

The expressed polypeptides have a N-glycan content comprising:
(i) 36-61% of the asialo-, agalacto-biantennary type; preferably 47-57%; more preferably 50-56.5%; most preferably 54.5-47.5%; and
(ii) 25.5-36.5% of the asialo-, mono-galactosylated-biantennary type and has a core substituted with fucose; preferably 30-35%; more preferably 31-34.5%; most preferably 32-34%; and
(iii) 5-11.5% of the asialo-, galactosylated biantennary type; preferably 5.1-10%; more preferably 5.2-9%; most preferably 5.3-8.7%; and
(iv) 0.8-3.5 of the oligomannose 5 and oligomannose 6 type; preferably 1.0-3.2%; more preferably 1.1-3.1%; most preferably 1.5-3.0%;
   as determined using high-performance hydrophilic interaction liquid chromatography with fluorescence detection (HILIC).

In an alternative preferred embodiment, said expressed polypeptides have a N-glycan content comprising
(i) 36.5-60% of the asialo-, agalactosylated-biantennary type and having a core substituted with fucose; preferably 40-58%; more preferably 45-56%; most preferably 47-54%; and
(ii) 24.5-37.5% of the asialo-, mono-galactosylated-biantennary type and has a core substituted with fucose and without a bisecting N-acetylglucosamin; preferably 25.5-37%; more preferably 27.5-35%; most preferably 30-34%; and
(iii) 3.5-10.5% of the asialo-, galactosylated-biantennary type has a core substituted with fucose and without a bisecting N-acetylglucosamin; preferably 5-9%; more preferably 6-8.5%; most preferably 6.5-8.2%; and
(iv) 0.5-3.1 of the oligomannose 5 type; preferably 0.6-2.9%; more preferably 0.7-2.5%; most preferably 0.9-2.0%;
   as determined using high-performance hydrophilic interaction liquid chromatography with fluorescence detection (HILIC).

The cell culture of this disclosure has a peak viable cell concentration of more than 1.2×10⁶ cells/ml, and achieves a productivity of more than 3.0 g/l.

In addition, the present invention provides a method for producing a therapeutic monoclonal antibody, in particular natalizumab, comprising the steps of
(a) cultivating a cell culture according to the present invention; and
(b) recovering the polypeptide comprising amino acids 19 to 231 of SEQ ID NO: 2 and the polypeptide comprising amino acids 19 to 468 of SEQ ID NO: 4 from said cell culture.

Finally, the present invention also provides a cell of the cell culture of the invention, and the use of said cell of the cell culture of the invention in the production of a therapeutic antibody, in particular wherein the therapeutic antibody is natalizumab.

### DETAILED DESCRIPTION OF THE PREFERRED EMBODIMENTS

More specifically, the present disclosure provides a cell culture obtainable from CHO DG44 cells which are capable of being cultured under serum-free or protein-free culture conditions, and which express a polypeptide comprising amino acids 19 to 231 of SEQ ID NO: 2 and a polypeptide comprising amino acids 19 to 468 of SEQ ID NO: 4.

The cell culture of the disclosure is obtainable from CHO DG44 cells, e.g. by using a suitable screening and subculturing approach as reported in Example 1 herein. CHO DG44 cells are commercially available, and characterized in that they are DHFR negative. The cells of the cell culture of the present disclosure have been adapted to serum-free and protein-free cell culture conditions. In the present case, this was achieved by gradually reducing serum concentrations from 10% to 2% to 0.5% to 0.1% and to 0%. Cells which have been adapted to serum-free conditions can also be cultured in protein-free media. Suitable media for protein-free cell culture of CHO cells are also commercially available. In a preferred embodiment, the cells express a polypeptide comprising the amino acid sequence of SEQ ID NO: 2 and a polypeptide comprising the amino acid sequence of SEQ ID NO: 4, i.e. the expressed polypeptide comprises the signal sequence shown in positions 1-18 of SEQ ID NO: 2 and SEQ ID NO: 4, respectively. Generally, the antibody may comprise a further tag or fusion, which can alleviate purification of the antibody. However, since any such tag could increase the antigenicity of the antibody, it is more preferred that the cells express a polypeptide consisting of the amino acid sequence of SEQ ID NO: 2, and a polypeptide consisting of SEQ ID NO: 4.

As demonstrated in Examples 3 and 4 below, said expressed polypeptides have a N-glycan content comprising:
(i) 36-61% of the asialo-, agalacto-biantennary type; preferably 47-57%; more preferably 50-56.5%; most preferably 54.5-47.5%; and
(ii) 25.5-36.5% of the asialo-, mono-galactosylated-biantennary type and has a core substituted with fucose; preferably 30-35%; more preferably 31-34.5%; most preferably 32-34%; and
(iii) 5-11.5% of the asialo-, galactosylated biantennary type; preferably 5.1-10%; more preferably 5.2-9%; most preferably 5.3-8.7%; and
(iv) 0.8-3.5 of the oligomannose 5 and oligomannose 6 type; preferably 1.0-3.2%; more preferably 1.1-3.1%; most preferably 1.5-3.0%;
   as determined using high-performance hydrophilic interaction liquid chromatography (HILIC) with fluorescence detection. See also Tables 3a and 3b below. As used herein and as referred to in the claims, the N-glycan content is determined by analyzing glycans enzymatically released from the protein that are labeled with a fluorescent molecule (ex. 2-aminobenzamide; 2-AB), followed by high-performance hydrophilic interaction liquid chromatography with fluorescence detection (HILIC).

In a preferred embodiment, labeling is carried out with 2AB using the following protocol:
1) Denaturation and deglycosylation—in this step we use RapiGest SF (Waters, P/N 186002123) for denaturation, DDT for reduction, iodoacetamide (IAM) for alkylation and for deglycosylation PGNase F (New England Biolab, cat. No. P0704S). Briefly 100 μg glycoprotein sample are dissolved in 50 mM ammonium bicarbonate (Sigma Aldrich Cat No. 09830-500G) to a final concentration of about 1 mg/ml. The standard is treated in the same way. 50 μl RapiGest of 0.5% RapiGest solution is added to the solution, and the reaction is incubated for 10 min at 22° C. Then, 4 μl of 0.5 M DTT is added to the samples, following incubation for 30 min at 37° C. Following this second incubation, 4 μl of 0.5 M IAM is added to the samples, and it is again incubated for 30 min at 22° C. in the dark. Finally, 1 μl of 10 mU/μl of PNGase F solution is added to each sample, and it is incubated overnight (about 18 h) at 37° C.
2) Extraction of released glycans—for extraction of released glycans the GlycoWorks HILIC μElution plate is used. After extraction of released glycans the formic acid treatment step is performing. In this step low concentration of formic acid is used in purpose to convert all glycans to free reducing glycans, hence improving the overall yield of the FLR-labeling via reductive animation. In brief, 250 μl acetonitrile (Sigma Aldrich Cat No. 360457-1L) is added to the reaction mixture obtained in step 1). The GlycoWorks HILIC μElution plate is conditioned by first adding 200 μl Mili-Q water and aspirating using the vacuum manifold, and then 200 μl of 85% acetonitrile followed by aspiration. Then the samples are loaded and it is washed three times with each 200 μl of 85% acetonitrile. The waste tray is then replaced with a 96-well collection plate with glass inserts. The glycans are eluted two times with each 100 μl of 100 ammonium acetate in 5% acetonitrile. The eluates are transferred into a new Eppendorf tube, and 100 μl of 1% formic acid solution is added to each sample, followed by incubation for 40 minutes at 22° C. Subsequently, the glycans are dried using vacuum evaporation bringing them to complete dryness. It is essential to have the sample completely dry before proceeding to the next step.
3) FLR labeling reaction of glycans—for labeling is using mixture of acetic acid, DMSO, 2-AB and sodium cyanoborohydride. In short, the labeling mixture is prepared by mixing 300 μl acetic acid with 700 μl DMSO and 10 mg 2AB. The entire contents is added to the vial of sodium cyanoborohydride in the GlycoWorks reagent kit (Waters, Cat. No. 186007034). The mixture should be protected from light and be used within an hour. 10 μl of the labeling solution is added to each dried sample ensuring that thew glycans are fully reconstituted in the 2AB label. Then the samples are incubated for 3.5 h at 65° C. under protection from light.
4) Excess labeling reagent removal—for this purpose a new well of the Glycoworks HILIC μElution plate is used. More specifically, 100 μl acetonitrile is added to 10 μl of 2AB labeled glycan sample. The mixture is then loaded on and eluted from the Glycoworks HILIC μElution plate using the same conditions as set out in step 2 above. The glycans can then be dried by evaporation. Glycans can be stored in ultrapure water at −20° C. until required.

The labeled 2-AB-glycan composition is then separated and determined by HILIC-UPLC measurement. The chromatographic separation is carried out by Cation Exchange High Performance Liquid Chromatography on UPLC H-Class Bio System using fluorescent detection (excitation at 330 nm and emission at 420 nm) under Empower™ Software control. The Waters BEH Glycan (1.7 μm, 4.6 mm i.d.×150 mm) is used applying eluents: A: Acetonitrile, and B: 0.1M Ammonium formate adjusted to pH 4.4 with formic acid. The glycans are separated using a linear gradient from 22% B to 44.1% B in 38.5 min with flow rate 0.7 ml/min and the column temperature is 60° C. Gradient is followed by a washing step of 100% eluent B in 2 min and re-equilibration with 78% solvent A. The total run time is 48 min. Data is evaluated using Waters Empower 3 software. The peak assignment is performed by retention time. The sample composition was determined by detecting peaks based on their retention time and the relative proportions of each peak were calculated from the peak areas, as also shown in FIGS. 3a and 3b.

Preferably, 36.5-60% of the asialo-, agalactosylated-biantennary type has a core substituted with fucose (G0F); more preferably 40-58%; even more preferably 45-56%; and most preferably 47-54%. It is also preferred that 0.3-0.9% of the asialo-, agalactosylated-biantennary type has a core substituted with fucose and has a bisecting N-acetylglucosamin (G0FB); more preferably 0.35-0.85%; even more preferably 0.45-0.8%; and most preferably 0.4-0.75%. Further, it is preferred that 0.05-0.48 of the asialo-, mono-galactosylated-biantennary type which has a core substituted with fucose has a bisecting N-acetylglucosamin (G1FB); more preferably 0.1-0.47%; even more preferably 0.15-0.46%; and most preferably 0.17-0.45%. Likewise, it is preferred that 4.9-11% of the asialo-, galactosylated-biantennary type has a core substituted with fucose (G2F); more preferably 5-9%; even more preferably 5.1-8.5%; and most preferably 5.2-8.2%. Preferably, 0.05-0.5% of the asialo-, galactosylated-biantennary type has a core substituted with fucose and has a bisecting N-acetylglucosamin (G2FB); more preferably 0.1-0.45%; even more preferably 0.15-0.4%; and most preferably 0.17-0.35%. In addition, said expressed polypeptides are characterized in that they have a N-glycan content comprising 0.5-3.1 of the oligomannose 5 type; preferably 0.6-2.9%; more preferably 0.7-2.5%; most preferably 0.9-2.0%; and/or 0.1-0.35 of the oligomannose 6 type; preferably 0.11-0.3%; more preferably 0.12-0.25%; most preferably 0.13-0.2%.

Alternatively, or in addition, said expressed polypeptides have a N-glycan content comprising
(i) 36.5-60% of the asialo-, agalactosylated-biantennary type and having a core substituted with fucose; preferably 40-58%; more preferably 45-56%; most preferably 47-54%; and
(ii) 24.5-37.5% of the asialo-, mono-galactosylated-biantennary type and has a core substituted with fucose and without a bisecting N-acetylglucosamin; preferably 25.5-37%; more preferably 27.5-35%; most preferably 30-34%; and
(iii) 3.5-10.5% of the asialo-, galactosylated-biantennary type has a core substituted with fucose and without a bisecting N-acetylglucosamin; preferably 5-9%; more preferably 6-8.5%; most preferably 6.5-8.2%; and
(iv) 0.5-3.1 of the oligomannose 5 type; preferably 0.6-2.9%; more preferably 0.7-2.5%; most preferably 0.9-2.0%;
   as determined using high-performance hydrophilic interaction liquid chromatography with fluorescence detection (HILIC), as described in detail above and in Example 4.

Interestingly, the cell culture of the present disclosure is not only characterized in that the produced antibody has a similar glycosylation pattern, but at the same time the cell culture exhibits a high peak viable cell concentration, and an improved productivity as compared to the NS0 expression system.

As used herein, the term “the peak viable cell concentration” is intended to mean the peak viable cell concentration as determined using trypan blue staining. In a preferred embodiment, a Vi-Cell XR Viability Analyzer is used for this determination. The Vi-CELL XR Cell Viability Analyzer is a video imaging system for analyzing yeast, insect and mammalian cells in culture media or in suspension. It automates the trypan blue dye exclusion protocol and is designed to analyze a wide variety of cell types. The software includes features to monitor bioreactors and other cell culture processes and is designed to comply with the US Food and Drug Administration's (FDA) regulations on electronic records and electronic signatures (21 CFR Part 11). Vi-CELL XR Cell Viability Analyzer works in concentration range of 50,000 to 10,000,000 cells per mL and the cell size range of 2 μm to 70 μm. The measurement of overall health of cell cultures requires accurate measurements of both cell concentration and percentage of viable or live cells.

The trypan blue dye exclusion method is a generally known method for cell viability determination. When cells die, their membranes become permeable allowing for the uptake of the trypan blue dye. As a result, the dead or non-viable cells become darker than the viable cells. This contrast is measured in order to determine viability. The Beckman Coulter Vi-CELL XR automates the Trypan Blue Dye Exclusion Method. Utilizing video capture technology and sample handling, the Vi-CELL XR takes the cell sample and delivers it to a flow cell and camera for imaging. The Vi-CELL XR will then capture up to 100 images for its determination of cellular viability. The software determines which cells have absorbed trypan blue dye and those that have not. Cells absorbing the trypan blue dye appear darker hence have lower gray scale values. Cells with higher gray scale values are considered viable. Briefly, the protocol includes the following steps:
1. Place a minimum of 0.5 mL (max. 2.5 mL) of sample into a sample cup.
2. Place sample cup in available carousel position.
3. Log in samples by clicking on the Log in sample button.
4. Select sample cup position on the carousel (if applicable).
5. Enter Sample ID.
6. Choose a Cell type.
7. Select Dilution factor if pre-diluted.
8. Click OK.
9. Press Start queue to begin the analysis.

Preferably, the peak viable cell concentration is more than 1.2×10⁶ cells/ml, such as more than 1.3×10⁶ cells/ml, more preferably more than 1.4×10⁶ cells/ml, more preferably more than 1.5×10⁶ cells/ml, more preferably more than 1.6×10⁶ cells/ml, more preferably more than 1.7×10⁶ cells/ml, and most preferably more than 1.8×10⁶ cells/ml.

It is understood by the artisan that an extended fermentation time may increase the productivity of a cell line in terms of the amount of the target protein which is obtained per volume of the cell culture, while a shorter fermentation time will typically result in a reduced yield. For sake of clarity, the term “productvity” as used herein is intended to mean the productivity of a 13 days process as determined using protein A chromatography. More specifically, UPLC measurements (UPLC system bio H-class) for the quantification of natalizumab in cell culture supernatants are performed using Protein-A HPLC. Briefly, cell culture supernatants were loaded onto a Protein A column (POROS A 20 2.1×30 mm, Applied Biosystems) with 50 mM sodium phosphate buffer 0.15 M NaCl pH 7.5 (mobile phase A) and bound Natalizumab was eluted by a shift to 50 mM sodium phosphate buffer 0.15 M NaCl pH 2.5 (mobile phase B). Wash and purge solution is 50 mM sodium phosphate buffer pH 7.5. Gradient started with pre-equilibration of 100% buffer A in 0.8 min, then 30% buffer B in 0.1 min was achieved. Elution linear gradient started from 30% to 100% of buffer B in 3.1 min. After elution the column was washed with 100% solvent B for 2 min and re-equilibrated with 100% solvent A. The total run time is 12 min. The flow rate was 0.5 ml/min, and the injection volume is 2 μl. The column temperature was 25° C. and elution is monitored at 280 nm. Product concentrations are determined by comparison with a standard curve, which is generated with reference material (natalizumab). The quantification method has a variation of about +10%.

The cell culture of the present disclosure achieves a productivity of more than 2.7 g/l; preferably more than 3.0 g/l; more preferably more than 3.5 g/l; more preferably more than 4.0 g/l, more preferably more than 4.1 g/l, more preferably more than 4.2 g/l; more preferably more than 4.3 g/l; more preferably more than 4.4 g/l; more preferably more than 4.5 g/l; more preferably more than 4.6 g/l; more preferably more than 4.7 g/l; and most preferably more than 4.8 g/l.

Moreover, the present disclosure provides a method for producing a therapeutic monoclonal antibody, in particular natalizumab, comprising the steps of
(a) cultivating a cell culture of the present disclosure; and
(b) recovering the polypeptide comprising amino acids 19 to 231 of SEQ ID NO: 2 and the polypeptide comprising amino acids 19 to 468 of SEQ ID NO: 4 from said cell culture.

Step (b) can be carried out using standard techniques as known in the field, including Protein A affinity chromatography, as described herein in further detail.

Likewise, the present disclosure further provides the use of a cell of the cell culture according to the present disclosure in the production of a therapeutic antibody, in particular natalizumab.

In a final aspect, the present disclosure also provides a cell of the cell culture according to the present disclosure.

The invention is further described by the following embodiments.
1. A cell culture obtainable from CHO DG44 cells which are capable of being cultured under serum-free or protein-free culture conditions, and which express a polypeptide comprising amino acids 19 to 231 of SEQ ID NO: 2 and a polypeptide comprising amino acids 19 to 468 of SEQ ID NO: 4.
2. The cell culture of embodiment 1, wherein the cells express a polypeptide comprising the amino acid sequence of SEQ ID NO: 2 and a polypeptide comprising the amino acid sequence of SEQ ID NO: 4.
3. The cell culture of embodiment 1, wherein the cells express a polypeptide consisting of the amino acid sequence of SEQ ID NO: 2 and a polypeptide consisting of SEQ ID NO: 4.
4. The cell culture of any one of embodiments 1 to 3, wherein the CHO DG44 host cells are DHFR negative.
5. The cell culture of any one of embodiments 1 to 4, wherein said expressed polypeptides have a N-glycan content comprising:
   (i) 36-61% of the asialo-, agalacto-biantennary type; preferably 47-57%; more preferably 50-56.5%; most preferably 54.5-47.5%; and
   (ii) 25.5-36.5% of the asialo-, mono-galactosylated-biantennary type and has a core substituted with fucose; preferably 30-35%; more preferably 31-34.5%; most preferably 32-34%; and
   (iii) 5-11.5% of the asialo-, galactosylated biantennary type; preferably 5.1-10%; more preferably 5.2-9%; most preferably 5.3-8.7%; and
   (iv) 0.8-3.5 of the oligomannose 5 and oligomannose 6 type; preferably 1.0-3.2%; more preferably 1.1-3.1%; most preferably 1.5-3.0%;
   as determined using high-performance hydrophilic interaction liquid chromatography with fluorescence detection (HILIC).
6. The cell culture of embodiment 5, wherein 36.5-60% of the asialo-, agalactosylated-biantennary type has a core substituted with fucose; preferably 40-58%; more preferably 45-56%; most preferably 47-54%.
7. The cell culture of embodiment 6, wherein 0.3-0.9% of the asialo-, agalactosylated-biantennary type has a core substituted with fucose and has a bisecting N-acetylglucosamin; preferably 0.35-0.85%; more preferably 0.45-0.8%; most preferably 0.4-0.75%.
8. The cell culture of any one of embodiments 5 to 7, wherein 0.05-0.48 of the asialo-, mono-galactosylated-biantennary type which has a core substituted with fucose has a bisecting N-acetylglucosamin; preferably 0.1-0.47%; more preferably 0.15-0.46%; most preferably 0.17-0.45%.
9. The cell culture of any one of embodiments 5 to 8, wherein 4.9-11% of the asialo-, galactosylated-biantennary type has a core substituted with fucose; preferably 5-9%; more preferably 5.1-8.5%; most preferably 5.2-8.2%.
10. The cell culture of embodiment 9, wherein 0.05-0.5% of the asialo-, galactosylated-biantennary type has a core substituted with fucose and has a bisecting N-acetylglucosamin; preferably 0.1-0.45%; more preferably 0.15-0.4%; most preferably 0.17-0.35%.
11. The cell culture of any one of embodiments 5 to 10, wherein said expressed polypeptides have a N-glycan content comprising
   0.5-3.1 of the oligomannose 5 type; preferably 0.6-2.9%; more preferably 0.7-2.5%; most preferably 0.9-2.0%; and/or
   0.1-0.35 of the oligomannose 6 type; preferably 0.11-0.3%; more preferably 0.12-0.25%; most preferably 0.13-0.2%.
12. The cell culture of any one of embodiments 1 to 11, wherein said expressed polypeptides have a N-glycan content comprising
(i) 36.5-60% of the asialo-, agalactosylated-biantennary type and having a core substituted with fucose; preferably 40-58%; more preferably 45-56%; most preferably 47-54%; and
(ii) 24.5-37.5% of the asialo-, mono-galactosylated-biantennary type and has a core substituted with fucose and without a bisecting N-acetylglucosamin; preferably 25.5-37%; more preferably 27.5-35%; most preferably 30-34%; and
(iii) 3.5-10.5% of the asialo-, galactosylated-biantennary type has a core substituted with fucose and without a bisecting N-acetylglucosamin; preferably 5-9%; more preferably 6-8.5%; most preferably 6.5-8.2%; and
(iv) 0.5-3.1 of the oligomannose 5 type; preferably 0.6-2.9%; more preferably 0.7-2.5%; most preferably 0.9-2.0%;
   as determined using high-performance hydrophilic interaction liquid chromatography (HILIC) with fluorescence detection.
13. The cell culture of any one of embodiments 1 to 12, wherein the peak viable cell concentration is more than 1.2×10⁶ cells/ml, preferably more than 1.3×10⁶ cells/ml, more preferably more than 1.4×10⁶ cells/ml, more preferably more than 1.5×10⁶ cells/ml, more preferably more than 1.6×10⁶ cells/ml, more preferably more than 1.7×10⁶ cells/ml, and most preferably more than 1.8×10⁶ cells/ml.
14. The cell culture of any one of embodiments 1 to 13, wherein the cell culture achieves a productivity of more than 2 g/l; preferably more than 2.5 g/l; such as more than 2.7 g/L; more preferably more than 3.0 g/l; more preferably more than 3.5 g/l; more preferably more than 4.0 g/l, more preferably more than 4.1 g/l, more preferably more than 4.2 g/l; more preferably more than 4.3 g/l; more preferably more than 4.4 g/l; more preferably more than 4.5 g/l; more preferably more than 4.6 g/l; more preferably more than 4.7 g/l; and most preferably more than 4.8 g/l.
15. A cell of the cell culture according to any one of embodiments 1-14.
16. A method for producing a therapeutic monoclonal antibody, comprising the steps of
   (c) cultivating a cell culture according to any one of embodiments 1-14; and
   (d) recovering the polypeptide comprising amino acids 19 to 231 of SEQ ID NO: 2 and the polypeptide comprising amino acids 19 to 468 of SEQ ID NO: 4 from said cell culture.
17. The method of embodiment 16, wherein the therapeutic antibody is natalizumab.
18. Use of a cell of the cell culture according to any one of embodiments 1-14 in the production of a therapeutic antibody, in particular wherein the therapeutic antibody is natalizumab.

In the following, the present invention as defined in the claims is further illustrated by the following figures and examples, which are not intended to limit the scope of the present invention. All references cited herein are explicitly incorporated by reference.

### DESCRIPTION OF THE FIGURES

FIG. 1: Exemplary chromatogram of test solution for titer assay. It shows a single peak for natalizumab at 4.424 minutes.

FIG. 2a: Exemplary chromatogram of standard solution for charge variants content analysis. It shows one main peak “PM” at 22.426 minutes, and two smaller peaks, “AS1” 20.682 minutes, and “BS1” 23.972 minutes, and a very low and broad peak “BS4” 36.578 minutes.

FIG. 2b: Exemplary chromatogram of tested solution for charge variants content analysis. It shows one main peak “PM” at 20.268 minutes, and two smaller peaks, “AS1” 18.067 minutes, and “BS1” 21.783 minutes, and three minor and broad peaks “BS2” 27.304 minutes, “BS3” 31.776 minutes, and “BS4” 36.578 minutes.

FIG. 3a: Exemplary chromatogram of solution for peak identification in N-glycoprofiling test. The Indicated peaks are (in the order of appearance): “peak 2” 11.386 minutes; “NGA2/G0” 11.965 minutes; “NGA2F/G0F” 13.550 minutes; “Man-5” 14.372 minutes; “NGA2FB/G0FB” 14.939 minutes; “Unkn.2 (Std.Mix)/G1 (Std.WAT)” 15.350 minutes; “Unkn. 3 (Std.Mix)” 16.118 minutes; “NA2G1F/G1F” 16.417 minutes; “NA2G1F/G1F*” 16.854 minutes; “NA2G1FB/G1FB” 17.446 minutes; “Man-6” 17.686 minutes; “NA2/G2” 18.149 minutes, “NA2F/G2F” 19.491 minutes; “NA2FB/G2FB” 20.154 minutes; “G1FS1” 21.585 minutes; “Unkn. 5 (Std.Mix)” 22.674 minutes; “A1F” 23.842 minutes; “Unkn. 6 (Std.Mix)” 24.655 minutes; “Unkn. 7 (Std.Mix)” 26.617 minutes; “Unkn. 8 (Std.Mix)” 27.595 minutes; “Unkn. 9 (Std.Mix)” 28.030 minutes.

FIG. 3b: Exemplary chromatogram of reference product solution for N-glycoprofiling test. The indicated peaks are (in the order of appearance): 9.130; “peak 1” 9.708; 10.137; 10.343; “Unkn. 1 (Std.Mix) 10.844; 11.091; “peak 2” 11.419 minutes; “NGA2/G0” 11.970; “NGA2F/G0F” 13.514 minutes; “Man-5” 14.316 minutes; 14.671 minutes; “NGA2FB/G0FB” 14.871 minutes; “Unkn.2 (Std.Mix)/G1 (Std.WAT)” 15.235 minutes; “NA2G1F/G1F” 16.370 minutes; “NA2G1F/G1F*” 16.794 minutes; “Man-6” 17.664 minutes; 17.880; “NA2/G2” 18.226 minutes, “NA2F/G2F” 19.484 minutes; 19.902 minutes; “NA2FB/G2FB” 20.236 minutes; 20.723; “Unkn. 4 (Std.Mix)” 21.068 minutes; “G1FS1” 21.632 minutes; 22.192; 22.595; “Unkn. 5 (Std.Mix)” 22.973 minutes; 23.296; “A1F” 24.034 minutes; “Unkn. 6 (Std.Mix)” 24.724 minutes; “peak 4” 25.105; 25.998; 27.341; 29.949.

FIG. 4a: Exemplary chromatogram of RapiFluor-MS Glycan Performance Test Standard solution (GPTS). The indicated peaks are (in the order of appearance): “G0” 11.887 minutes; “G0F” 12.900 minutes; “G0FB” 14.024 minutes; “A2G1” 14.268 minutes; “A2G1′” 14.637 minutes; “G1F” 15.215 minutes; “G1F′” 15.611 minutes; “G1FB” 16.075 minutes; “G1FB” 16.414 minutes; “A2G2” 16.931 minutes; “G2F” 17.809 minutes; “G2FB” 18.354 minutes, “G1FS1” 19.065 minutes; 19.986 minutes; 20.226 minutes; “A1F” 20.975 minutes; “FA2BG2S1” 21.672 minutes; 22.727 minutes; 23.106 minutes; 23.586 minutes; “FA2G2S2/A2F” 23.763 minutes; “FA2BG2S2” 24.165 minutes; 25.428 minutes.

FIG. 4b: Exemplary chromatogram of Ribonuclease B solution. The indicated peaks are (in the order of appearance): 13.805 minutes; “Man-5” 14.073 minutes; 15.579 minutes; “Man-6′” 16.567 minutes; 18.128 minutes; “Man-7” 19.044 minutes; “Man-7′” 19.435 minutes; 21.338 minutes; 21.500 minutes; “Man-8” 21.771 minutes; “Man-9” 23.566 minutes.

FIG. 4c: Exemplary chromatogram of reference product solution. The indicated peaks are (in the order of appearance): 8.395 minutes; 9.567 minutes; 10.184 minutes; 11.009 minutes; 11.329 minutes; 11.558 minutes; 11.677 minutes; “G0” 11.886 minutes; 12.267 minutes; 12.619 minutes; “G0F” 12.904 minutes; 13.443 minutes, 13.530 minutes; 13.685 minutes; 13.846 minutes; “Man-5” 14.116 minutes; 14.432 minutes; “A2G1” 14.648 minutes; “G1F” 15.217 minutes, “G1F” 15.613 minutes; 16.097 minutes; “G1FB” 16.283 minutes; “G1FB” 16.617 minutes; “Man-6/A2G2 (?)” 16.929 minutes; “G2F” 17.809 minutes; 18.181 minutes; 18.668 minutes, “G1FS1” 19.012 minutes; 19.276 minutes; 19.786 minutes; 20.087 minutes; 20.031 minutes; “A1F” 20.911 minutes; 21,268 minutes; 21.992 minutes; 22.158 minutes; 22.313 minutes; 22.655 minutes; 22.968 minutes; 23.331 minutes; 24.060 minutes; 24.550 minutes; 25.904 minutes.

### DESCRIPTION OF THE SEQUENCES

The start and stop codons are indicated in bold, the signal sequence is underlined.

### Natalizumab Light Chain

(SEQ ID NO: 1)
Atgaagtgggtgaccttcatctccctgctgtttctgttctcctccgcctac
tccgacatccagatgacccagtccccctccagcctgtccgcctccgtgggc
gacagagtgaccatcacatgcaagacctcccaggacatcaacaagtacatg
gcctggtatcagcagacccccggcaaggcccctcggctgctgatccactac
acctccgccctgcagcccggcatcccttccagattctccggctctggctct
ggccgggactacaccttcaccatctccagcctgcagcctgaggacattgcc
acctactactgcctgcagtacgacaacctgtggaccttcggccagggcacc
aaggtggaaatcaagcggaccgtggccgctccctccgtgttcatcttccca
ccctccgacgagcagctgaagtccggcaccgccagcgtggtgtgcctgctg
aacaacttctacccccgcgaggccaaggtgcagtggaaggtggacaacgcc
ctgcagagcggcaactcccaggaatccgtgaccgagcaggactccaaggac
agcacctactccctgtcctccaccctgaccctgtccaaggccgactacgag
aagcacaaggtgtacgcctgcgaagtgacccaccagggcctgtccagcccc
gtgaccaagtccttcaaccggggcgagtgctgatag
Translation
(SEQ ID NO: 2)
MKWVTFISLLFLFSSAYSDIQMTQSPSSLSASVGDRVTITCKTSQDINKYM
AWYQQTPGKAPRLLIHYTSALQPGIPSRFSGSGSGRDYTFTISSLQPEDIA
TYYCLQYDNLWTFGQGTKVEIKRTVAAPSVFIFPPSDEQLKSGTASVVCLL
NNFYPREAKVQWKVDNALQSGNSQESVTEQDSKDSTYSLSSTLTLSKADYE
KHKVYACEVTHQGLSSPVTKSFNRGEC

### Natalizumab Heavy Chain

(SEQ ID NO: 3)
atgaagtgggtgaccttcatctccctgctgtttctgttctccagcgcctac
tcccaggtgcagctggtgcagtctggcgccgaagtgaagaaacctggcgcc
tccgtgaaggtgtcctgcaaggcctccggcttcaacatcaaggacacctac
atccactgggtgcgacaggcccctggccagcggctggaatggatgggcaga
atcgaccccgccaacggctacactaagtacgaccccaagttccagggcaga
gtgaccatcaccgccgacacctccgcctccaccgcctacatggaactgtcc
tccctgcggagcgaggacaccgccgtgtactactgcgccagagagggctac
tacggcaactacggcgtgtacgccatggactactggggccagggcaccctg
gtgacagtgtcctccgccagcaccaagggcccctccgtgttccctctggcc
ccttgctcccggtccacctccgagtctaccgccgctctgggctgcctggtg
aaagactacttccccgagcccgtgaccgtgtcctggaactctggcgccctg
acctccggcgtgcacaccttccctqccgtgctgcagtcctccggcctgtac
tccctgtcctccgtggtgaccgtgccatccagctccctgggcaccaagacc
tacacctgtaacgtggaccacaagccctccaacaccaaggtggacaagcgg
gtggaatctaagtacggccctccctgccccagctgccctgcccctgaattc
ctgggcggaccttccgtgttcctgttccccccaaagcccaaggacaccctg
atgatctcccggacccccgaagtgacctgcgtggtggtggacgtgtcccag
gaagatcccgaggtgcagttcaattggtacgtggacggcgtggaagtgcac
aacgccaagaccaagcccagagaggaacagttcaactccacctaccgggtg
gtgtctgtgctgaccgtgctgcaccaggactggctgaacggcaaagagtac
aagtgcaaggtgtccaacaagggcctgcccagctccatcgaaaagaccatc
tccaaggccaagggccagccccgcgagccccaggtgtacaccctgccccct
agccaggaagagatgaccaagaaccaggtgtccctgacctgtctggtgaaa
ggcttctacccctccgacattgccgtggaatgggagtccaacggccagccc
gagaacaactacaagaccaccccccctgtgctggactccgacggctccttc
ttcctgtactctcggctgaccgtggacaagtcccggtggcaggaaggcaac
gtgttctcctgctccgtgatgcacgaggccctgcacaaccactacacccag
aagtccctgtccctgagcctgggcaagtgatag
Translation
(SEQ ID NO: 4)
MKWVTFISLLFLESSAYSQVQLVQSGAEVKKPGASVKVSCKASGFNIKDTY
THWVRQAPGQRLEWMGRIDPANGYTKYDPKFQGRVTITADTSASTAYMELS
SLRSEDTAVYYCAREGYYGNYGVYAMDYWGQGTLVTVSSASTKGPSVFPLA
PCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPEF
LGGPSVFLEPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQ
KSLSLSLGK

### EXAMPLES

### Example 1: Cell Line Development

An expression construct was generated based on a standard expression vector. The vector comprises two expression cassettes encoding the light and heavy chain of natalizumab, respectively. See also SEQ ID NO: 2 and SEQ ID NO: 4 above. The plasmid further contains a dihydrofolate reductase gene as a selection marker. Cloning of the expression vector was performed using molecular biological standard techniques. Plasmid DNA was prepared and verified by transforming competent E. coli cells and preparation of mini prep DNA (PureLink HiPure Plasmid Filter Maxiprep Kit) from a correct clone which was obtained during the molecular cloning procedure. Verification was by both restriction analysis and sequencing.

This expression construct was linearized, purified and concentrated by isopropanol precipitation, and used to transfect CHO DG44 host cell line using routine electroporation techniques.

The cells were subjected to selection and methotrexate (MTX) amplification procedures employing large pools (LPs), and mini pools (MPs). Briefly, following transfection, cells were cultivated in host cell growth medium for 2 days. Subsequently, they were transferred into selective medium and subcultivated in the same medium every 3-4 day until viability recovered and cells started to grow. Growing cells were transferred into selective medium+5 nm MTX, and subcultivated in the same medium every 3-4 day until viability recovered and cells started to grow. Subsequently, pools were transferred into selective medium+30 nM MTX to induce the amplification process and expanded up to shake flask level.

Subsequently, single cell clones were isolated from the best available cell pools by FACS sorting. Briefly, 3×10⁶ cells were centrifuged and stained with fluorescence conjugated Protein A. Subsequently, cells were washed, resuspended in PBS, filtered through a FACS tube with cell strainer cap and analyzed by flow cytometry. The top 3-5% population with regard to fluorescence was selected and single cells were sorted into 384 well flat bottom plates containing.

The best pools were chosen based on similarity of their glycan profile with the originator molecule, which were analysed from pool fed-batch supernatants, as further described in Examples 3 and 4 below. Subsequently, growing clones were pre-selected according to productivity as well as monoclonality and expanded up to shake flask level.

The best 40 high-expressing clones were chosen and evaluated in a standard fed-batch process with regard to productivity and process characteristics. The five best performing clones regarding product quality with the productivity between 2.1-4.4 g/L in 10 days process, were chosen as preferential production cell lines. For each of these clones, research cell banks consisting of 20 vials each were prepared and stored in the gas phase of liquid nitrogen. Subsequently, one vial of each clone was thawed and subjected to a stability study for 7 weeks including two fed-batch runs starting at different points in time of the study. The obtained data indicate that all clones are phenotypically stable. One of the clones, has a duration of 13 days, leads to peak viable cell concentrations of approximately 18.4×10⁶/mL and product concentrations of about 4.9 g/L.

### Example 2: Monoclonal Antibody (mAb) Concentration (Titer) Analysis

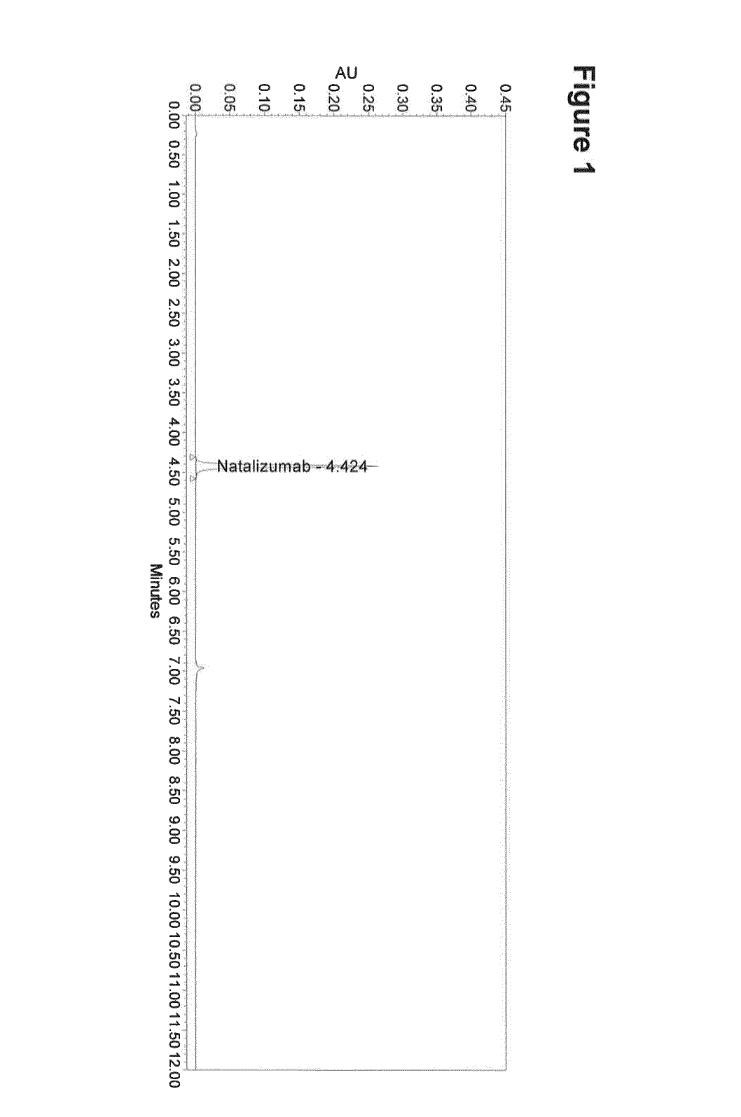

Chromatographic analysis by Protein A affinity chromatography was carried out on UPLC H-Class Bio System using UV detection under Empower 3 Software control. The Applied Biosystems Poros® Protein A column (20 μm, 2.1 mm i.d.×30 mm) was used for testing applying a two steps of linear gradient of buffer A (50 mM sodium phosphate buffer pH 7.5, 0.15 M NaCl) and buffer B (50 mM sodium phosphate buffer pH 2.5, 0.15 M NaCl). Gradient started with pre-equilibration of 100% buffer A in 0.8 min, then 30% buffer B in 0.1 min was achieved. Elution linear gradient started from 30% to 100% of buffer B in 3.1 min. After elution the column was washed with 100% solvent B for 2 min and re-equilibrated with 100% solvent A. The total run time is 12 min. The flow rate was 0.5 ml/min. The column temperature was 25° C. and elution is monitored at 280 nm. Exemplary chromatogram of test solution is presented on FIG. 1.

Mab concentration calculation based on linear standard curve was determined by Empower 3 Software. Exemplary results of mAb concentration for reference product (range based on 9 batches testing) and tested product from clone selection step are presented in Table 1.

TABLE 1
MAb titer results

| | mAb Titer [mg/ml] | | | | | |
|---|---|---|---|---|---|---|
| | rep. | Average | | | | |
| Sample | No (n) | Results | min | max | SD | CV |
| Clone 1 | 3 | 5.813 | 5.809 | 5.818 | 0.004 | 0.076 |
| Clone 2 | 3 | 5.667 | 5.657 | 5.680 | 0.012 | 0.209 |
| Referent (9 batches) | 3 | 20.255 | 19.400 | 22.187 | 0.850 | 0.042 |
| [20 mg/ml] | | | | | | |

Clones 1 and 2 showed antibody titers of more than 5.6 mg/ml=5.6 g/L.

### Example 3: Charge Variants Content by Cation Exchange Liquid Chromatography

Monoclonal antibodies are subject to post-translational modifications or degradation at several independent sites. Such modifications may result in the presence of many different species in the final product. Monoclonal antibodies therefore display considerable heterogeneity that can be characterized by ion exchange liquid chromatography (IEX-LC).

The separation was carried out by Cation Exchange High Performance Liquid Chromatography on UPLC H-Class Bio System using UV detection under Empower™ Software control. The Waters Protein-Pak Hi Res SP (7 μm, 4.6 mm i.d.×100 mm) was used for testing applying a linear gradient of NaCl. Eluents were: buffer A (10 mM NaPi buffer pH 6.0) and buffer B (10 mM NaPi buffer pH 6.0, 0.125 M NaCl). Gradient starts with pre-equilibration of 100% buffer A in 5 min. Elution gradient starts from 10% to 30% of buffer B in 25 min min, followed by a washing step for 5 min at 30% B and re-equilibration with 90% solvent A. The total run time is 45 min. The flow rate was 0.7 ml/min. The column temperature was 40° C. and elution is monitored at 220 nm.

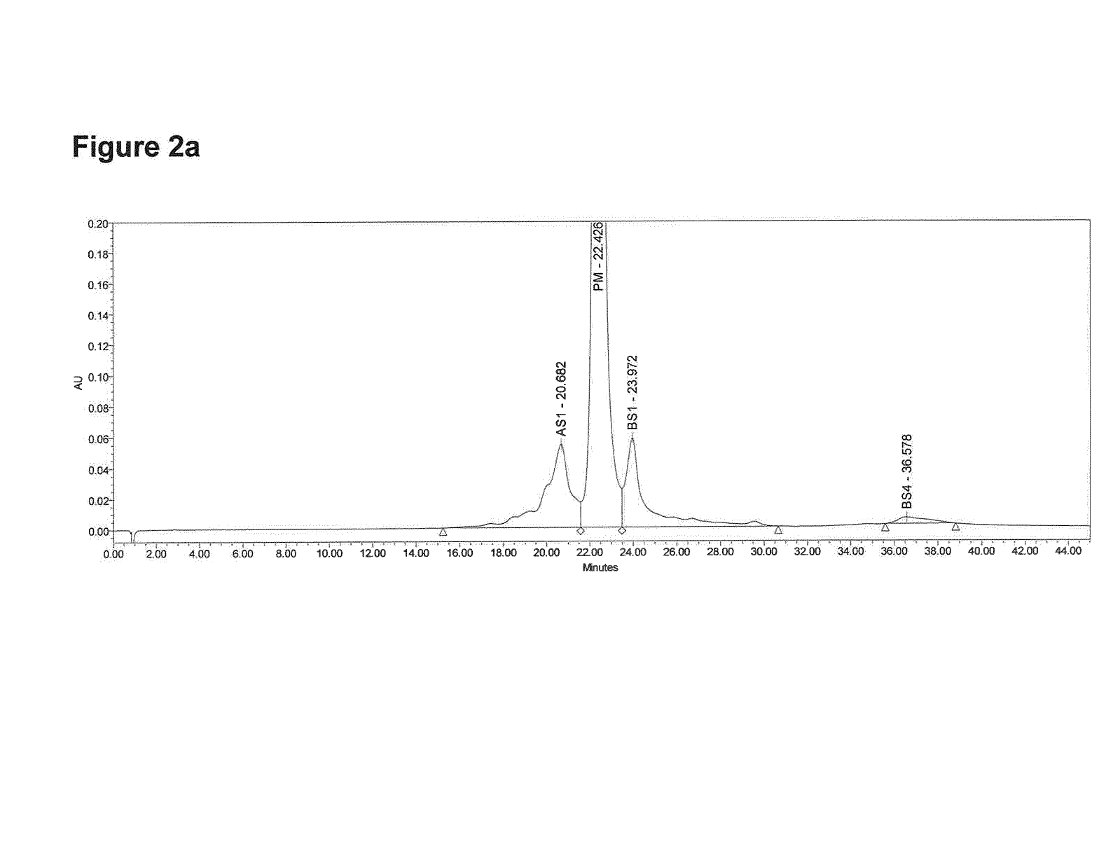

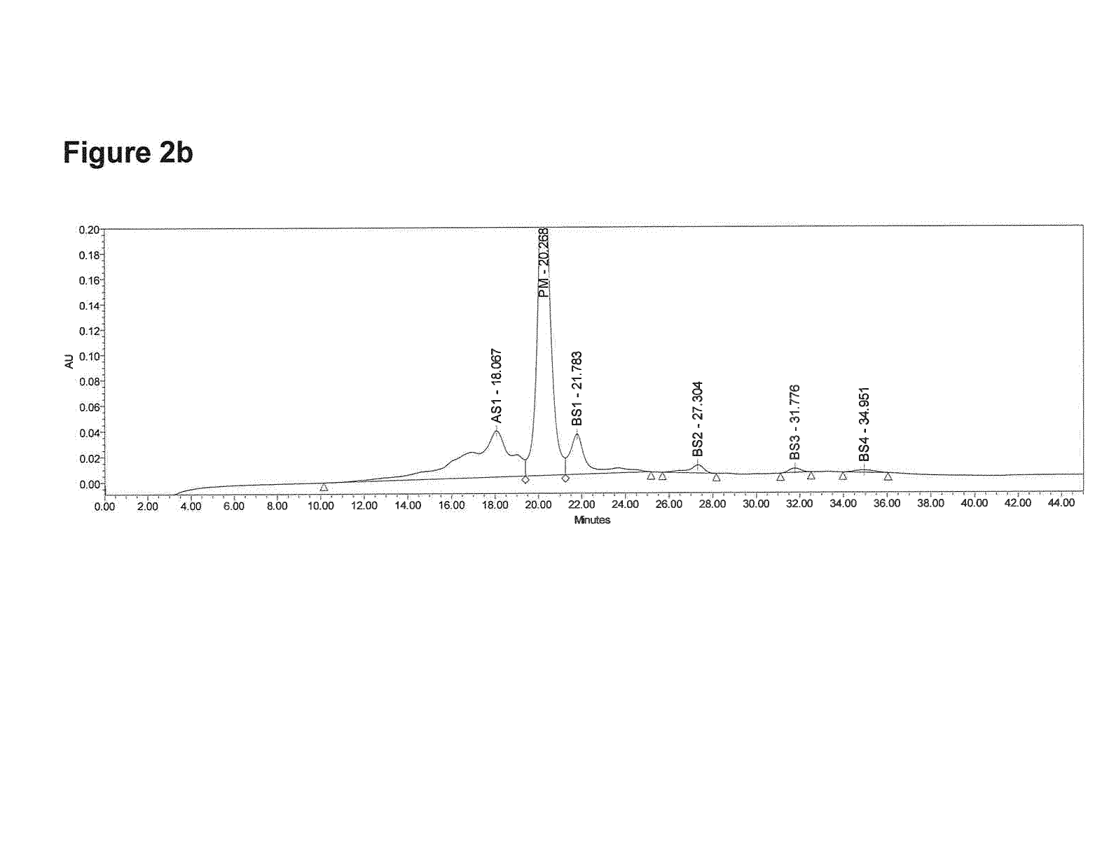

For data evaluation was used Waters Empower 3 software. The peak assignment was performed by retention time. The sample composition was determined by detecting peaks based on their retention time and the relative proportions of each peak were calculated from the peak areas. The final results were presented as a sum of acidic species, main peak and sum of basic species. Exemplary chromatograms of standard (reference) and test solution are presented on FIGS. 2a and 2b. Exemplary results of charge variants content for reference product (range based on 8 batches testing) and tested product from clone selection step are presented in Table 2.

TABLE 2
Charge Variants content results

| | CEX_MAIN PEAK | | | | | |
|---|---|---|---|---|---|---|
| | rep. | Average | | | | |
| Sample | No (n) | Results | min | max | SD | CV |
| Clone 1 | 3 | 44.80 | 44.30 | 45.08 | 0.432 | 0.96 |
| Clone 2 | 3 | 32.21 | 32.10 | 32.33 | 0.116 | 0.36 |
| Referent 9 batches) | 3 | 72.72 | 71.28 | 74.25 | 1.092 | 0.02 |
| | CEX_SUM of ACIDIC PEAKS | | | | | |
| | rep. | Average | | | | |
| Sample | No (n) | Results | min | max | SD | CV |
| Clone 1 | 3 | 41.26 | 41.15 | 41.42 | 0.142 | 0.34 |
| Clone 2 | 3 | 54.39 | 54.23 | 54.50 | 0.140 | 0.26 |
| Referent 9 batches) | 3 | 14.48 | 10.83 | 18.09 | 2.408 | 0.17 |
| | CEX_SUM of BASIC PEAKS | | | | | |
| | rep. | Average | | | | |
| Sample | No (n) | Results | min | max | SD | CV |
| Clone 1 | 3 | 13.937 | 13.56 | 14.54 | 0.528 | 3.79 |
| Clone 2 | 3 | 13.407 | 13.23 | 13.58 | 0.175 | 1.31 |
| Referent (9 batches) | 3 | 12.80 | 10.15 | 14.92 | 1.631 | 0.13 |

### Example 4: N-Glycosylation Profile Analysis with 2-Aminobenzamide Using Ultra Performance Liquid Chromatography with Fluorescence Detection

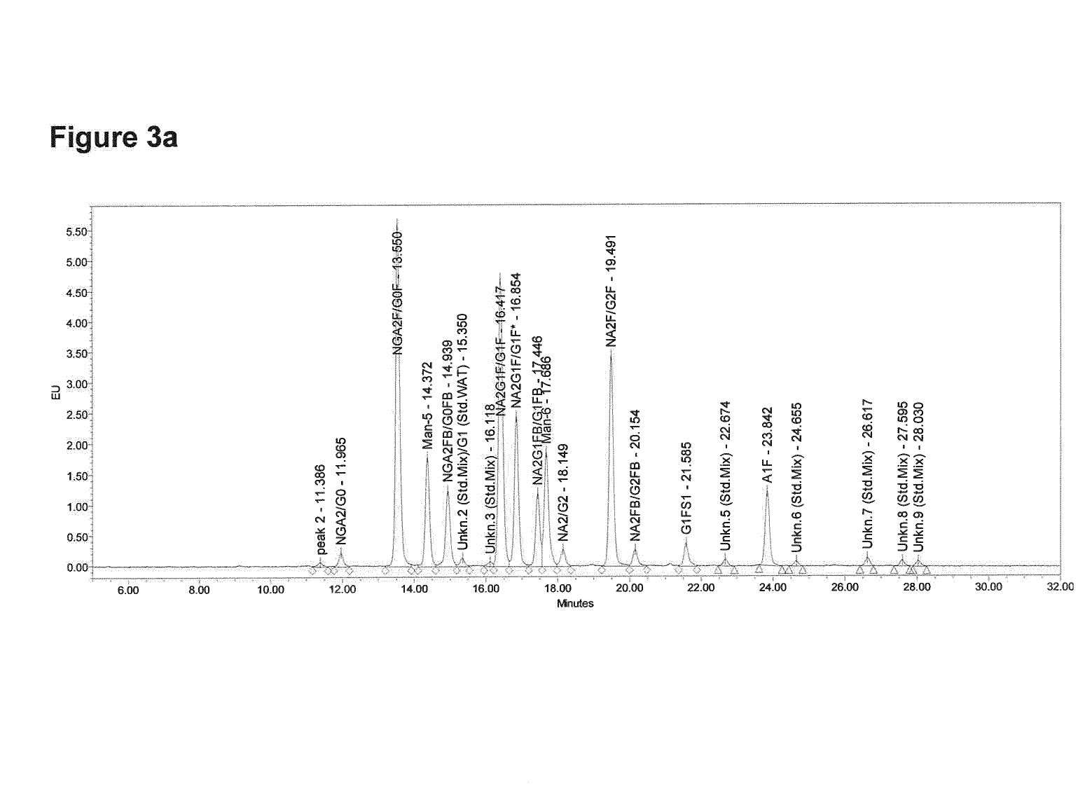

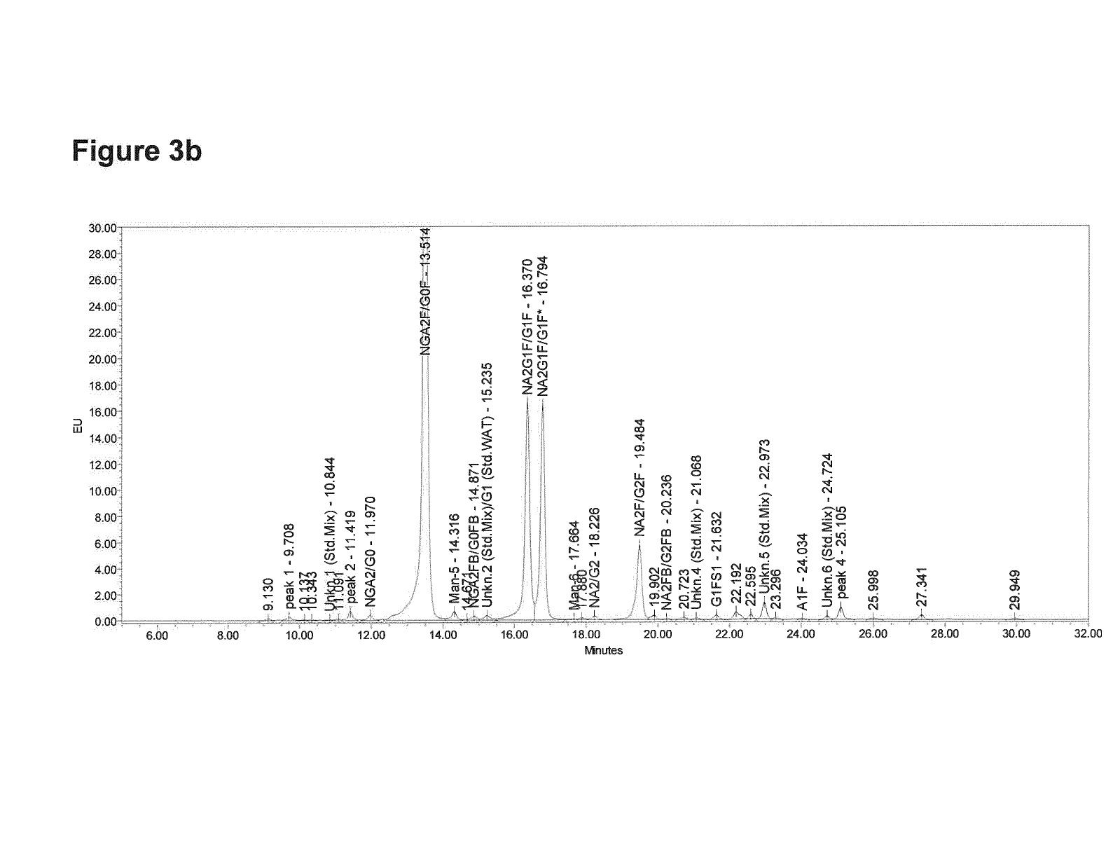

Glycosylation plays a predominant role in determining the function, pharmacokinetics, pharmacodynamics, stability, and immunogenicity of biotherapeutics. There are many physical functions of N-linked glycosylation in a mAb such as affecting its solubility and stability, protease resistance, binding to Fc receptors, cellular transport and circulatory half-life in vivo. Therefore, it is very important to quantitate and monitor the glycosylation pattern. The most common approach to qualitative and quantitative characterization of glycans is the analysis of glycans enzymatically released from the protein. This approach leads to mixtures of oligosaccharides that are label with a fluorescent molecule (ex. 2-aminobenzamide, 2-AB; Sigma Cat. No. A89804-100G), followed by high-performance hydrophilic interaction liquid chromatography with fluorescence detection (HILIC). The above described approach let us identify 13 of N-linked glycan's. The identification of glycans is performed by the order of elution and retention times of peaks from solution for peak identification (FIG. 3a). The names and structure of identified glycans were presented in Table 3a. Sample preparation protocol for the release of N-glycans consists of four steps:
5) Denaturation and deglycosylation—in this step we use RapiGest SF (Waters, P/N 186002123) for denaturation, DDT for reduction, iodoacetamide (IAM) for alkylation and for deglycosylation PGNase F (New England Biolab, cat. No. P0704S). Briefly 100 g glycoprotein sample are dissolved in 50 mM ammonium bicarbonate (Sigma Aldrich Cat No. 09830-500G) to a final concentration of about 1 mg/ml. The standard is treated in the same way. 50 μl RapiGest of 0.5% RapiGest solution is added to the solution, and the reaction is incubated for 10 min at 22° C. Then, 4 μl of 0.5 M DTT is added to the samples, following incubation for 30 min at 37° C. Following this second incubation, 4 μl of 0.5 M IAM is added to the samples, and it is again incubated for 30 min at 22° C. in the dark. Finally, 1 μl of 10 mU/μl of PNGase F solution is added to each sample, and it is incubated overnight (about 18 h) at 37′C.
6) Extraction of released glycans—for extraction of released glycans the GlycoWorks HILIC μElution plate is used. After extraction of released glycans the formic acid treatment step is performing. In this step low concentration of formic acid is used in purpose to convert all glycans to free reducing glycans, hence improving the overall yield of the FLR-labeling via reductive animation. In brief, 250 μl acetonitrile (Sigma Aldrich Cat No. 360457-1 L) is added to the reaction mixture obtained in step 1). The GlycoWorks HILIC μElution plate is conditioned by first adding 200 μl Mili-Q water and aspirating using the vacuum manifold, and then 200 μl of 85% acetonitrile followed by aspiration. Then the samples are loaded and it is washed three times with each 200 μl of 85% acetonitrile. The waste tray is then replaced with a 96-well collection plate with glass inserts. The glycans are eluted two times with each 100 μl of 100 ammonium acetate in 5% acetonitrile. The eluates are transferred into a new Eppendorf tube, and 100 μl of 1% formic acid solution is added to each sample, followed by incubation for 40 minutes at 22° C. Subsequently, the glycans are dried using vacuum evaporation bringing them to complete dryness. It is essential to have the sample completely dry before proceeding to the next step.
7) FLR labeling reaction of glycans—for labeling is using mixture of acetic acid, DMSO, 2-AB and sodium cyanoborohydride. In short, the labeling mixture is prepared by mixing 300 μl acetic acid with 700 μl DMSO and 10 mg 2AB. The entire contents is added to the vial of sodium cyanoborohydride in the GlycoWorks reagent kit (Waters, Cat. No. 186007034). The mixture should be protected from light and be used within an hour. 10 μl of the labeling solution is added to each dried sample ensuring that thew glycans are fully reconstituted in the 2AB label. Then the samples are incubated for 3.5 h at 65° C. under protection from light.
8) Excess labeling reagent removal—for this purpose a new well of the Glycoworks HILIC μElution plate is used. More specifically, 100 μl acetonitrile is added to 10 μl of 2AB labeled glycan sample. The mixture is then loaded on and eluted from the Glycoworks HILIC μElution plate using the same conditions as set out in step 2 above. The glycans can then be dried by evaporation. Glycans can be stored in ultrapure water at −20° C. until required.

The labeled 2-AB-glycan composition was separated and determined by HILIC-UPLC measurement. The chromatographic separation was carried out by Cation Exchange High Performance Liquid Chromatography on UPLC H-Class Bio System using fluorescent detection (excitation at 330 nm and emission at 420 nm) under Empower™ Software control. The Waters BEH Glycan (1.7 μm, 4.6 mm i.d.×150 mm) was used for testing applying eluents: A: Acetonitrile, and B: 0.1M Ammonium formate adjusted to pH 4.4 with formic acid. The glycans were separated using a linear gradient from 22% B to 44.1% B in 38.5 min with flow rate 0.7 ml/min and the column temperature was 60° C. Gradient was followed by a washing step of 100% eluent B in 2 min and re-equilibration with 78% solvent A. The total run time was 48 min.

For data evaluation was used Waters Empower 3 software. The peak assignment was performed by retention time. The sample composition was determined by detecting peaks based on their retention time and the relative proportions of each peak were calculated from the peak areas. The final results were presented as a sum of acidic species, main peak and sum of basic species. Exemplary chromatograms of standard (reference; GlycoWorks Control Standard, Waters, Cat no. 186007033) and test solution are presented on FIGS. 3a and 3b. Exemplary results of N-glycan content for reference product (range based on 9 batches testing) and tested product from clone selection step are presented in Table 3b.

TABLE 3b
N-glycan content results

| | rep. | | | | | | | | | | | | | | |
|---|---|---|---|---|---|---|---|---|---|---|---|---|---|---|---|
| Sample | No (n) | Glycans | G0 | G0F | G0FB | G1F | G1F′ | G1FB | Man-5 | Man-6 | G2 | G2F | G2FB | G1FS1 | A1F |
| Clone 1 | 3 | AV | 1.303 | 48.853 | 0.550 | 17.140 | 16.320 | 0.260 | 1.900 | 0.133 | 0.137 | 7.367 | 0.433 | 0.127 | 0.127 |
| | | min | 1.250 | 47.670 | 0.510 | 16.550 | 15.600 | 0.230 | 1.760 | 0.130 | 0.100 | 7.050 | 0.370 | 0.080 | 0.100 |
| | | max | 1.340 | 50.890 | 0.600 | 17.460 | 16.700 | 0.290 | 1.990 | 0.140 | 0.180 | 7.600 | 0.480 | 0.180 | 0.150 |
| | | sd | 0.047 | 1.772 | 0.046 | 0.512 | 0.624 | 0.030 | 0.123 | 0.006 | 0.040 | 0.284 | 0.057 | 0.050 | 0.025 |
| | | CV | 3.626 | 3.626 | 8.332 | 2.985 | 3.823 | 11.538 | 6.467 | 4.330 | 29.572 | 3.859 | 13.122 | 39.736 | 19.868 |
| Clone 2 | 3 | AV | 0.657 | 52.887 | 0.843 | 15.947 | 15.677 | 0.323 | 2.887 | 0.167 | 0.073 | 5.110 | 0.307 | 0.117 | 0.070 |
| | | min | 0.610 | 51.690 | 0.810 | 15.240 | 15.170 | 0.320 | 2.820 | 0.140 | 0.050 | 4.770 | 0.280 | 0.110 | 0.060 |
| | | max | 0.710 | 54.450 | 0.870 | 16.750 | 15.980 | 0.330 | 3.010 | 0.190 | 0.090 | 5.460 | 0.330 | 0.120 | 0.080 |
| | | sd | 0.050 | 1.416 | 0.031 | 0.760 | 0.442 | 0.006 | 0.107 | 0.025 | 0.021 | 0.345 | 0.025 | 0.006 | 0.010 |
| | | RSD | 7.665 | 2.678 | 3.623 | 4.764 | 2.817 | 1.786 | 3.704 | 15.100 | 28.386 | 6.754 | 8.206 | 4.949 | 14.286 |
| Referent | 3 | AV | 0.303 | 46.835 | 0.414 | 17.014 | 15.224 | 0.185 | 0.935 | 0.243 | 0.455 | 7.988 | 0.170 | 0.413 | 0.112 |
| (9 | | min | 0.133 | 36.553 | 0.307 | 14.187 | 11.663 | 0.097 | 0.540 | 0.127 | 0.313 | 4.910 | 0.067 | 0.233 | 0.047 |
| batches) | | max | 0.527 | 59.100 | 0.683 | 18.163 | 17.673 | 0.267 | 1.780 | 0.340 | 0.613 | 10.493 | 0.380 | 0.793 | 0.287 |
| | | sd | 0.135 | 7.499 | 0.145 | 1.394 | 2.412 | 0.063 | 0.524 | 0.069 | 0.117 | 1.953 | 0.119 | 0.222 | 0.080 |
| | | RSD | 0.445 | 0.160 | 0.351 | 0.082 | 0.158 | 0.340 | 0.560 | 0.283 | 0.257 | 0.245 | 0.701 | 0.538 | 0.717 |

### Example 5: Biological Activity Testing Details—Fab Related Activity

Antigen binding part of natalizumab (Fab) is responsible for the interaction with its antigen: α4 subunit of integrin. Mechanism of action for natalizumab involves blocking interaction of α4β1 and α4β7 integrins with their cognate receptors VCAM-1 and MadCAM-1, respectively. The comparability study is designed in a way to mimic the biological properties of natalizumab related to Fab functions.

### Integrin Binding by Direct ELISA

The aim of this assay is to confirm the potency of natalizumab to bind α4β1 integrin in a dose-dependent manner.

The principle of this method is to incubate a coated constant amount of integrin α4β1 with serially diluted natalizumab samples. The amount of bound natalizumab is subsequently determined by a mouse, monoclonal anti-human IgG antibody, which is conjugated to horseradish peroxidase (HRP). HRP converts the chromogenic substrate TMB (3,3′,5,5′-tetramethylbenzidine) into a colored dye. The color reaction is measured spectrophotometrically at wavelength 450 nm.

Data are analyzed applying 4 Parameter Logistic nonlinear regression model (4PL), which is commonly used for curve-fitting analysis in bioassays or immunoassays such as ELISAs or dose-response curves. Final result is expressed as a Relative Potency (REP) of tested sample in relation to interim reference standard established at Polpharma site. The method variability was determined at the level of 7% coefficient variation (CV) of intermediate precision within the qualification exercise.

TABLE 4
Integrin binding results by direct ELISA

| | Integrin Direct ELISA | | | | | |
|---|---|---|---|---|---|---|
| | rep. | Average | | | | |
| Sample | No (n) | Results | min | max | SD | CV |
| Clone 1 | 3 | 0.769 | 0.723 | 0.847 | 0.068 | 8.83 |
| Clone 2 | 3 | 0.679 | 0.611 | 0.732 | 0.062 | 9.13 |

The data in Table 4 shows that clones 1 and 2 bind to α4β1 integrin.

### VCAM-1 Competitive Binding by ELISA

The aim of this assay is to test the ability of natalizumab to inhibit interaction of α4β1 integrin with its cognate receptor—VCAM-1 protein in a dose-dependent manner.

Constant amount of the coated VCAM-1 protein is incubated with serial dilutions of natalizumab in the presence of HIS-tagged α4β1 integrin. Solid-phase associated VCAM-1 and soluble natalizumab now compete for binding to α4β1 integrin. The higher the natalizumab concentration the more α4β1 Integrin is inhibited from binding to VCAM-1. The highest signal result is observed when no natalizumab is present. Bound HIS-tagged α4β1 integrin is subsequently detected with a biotinylated anti-HIS-tag antibody, POD-conjugated Streptavidin and a TMB-substrate reaction at the end of the assay.

Data are analyzed with 4PL fitting model. Final result is expressed as a Relative Potency (REP) of tested sample in relation to reference standard. The method variability was determined at the level of 7% coefficient variation (CV) of intermediate precision within the qualification exercise. Additionally accuracy, linearity and specificity were tested.

TABLE 5
VCAM-1 binding results by competitive ELISA

| | VCAM-1 competitive ELISA | | | | | |
|---|---|---|---|---|---|---|
| | rep. | Average | | | | |
| Sample | No (n) | Results | min | max | SD | CV |
| Clone 1 | 3 | 0.829 | 0.805 | 0.842 | 0.021 | 2.54 |
| Clone 2 | 3 | 0.903 | 0.85 | 0.93 | 0.046 | 5.08 |
| Referent | 3 | 1.025 | 0.935 | 1.147 | 0.070 | 6.84 |
| (9 batches) | | | | | | |

Clones 1 and 2 show a similar ability of inhibiting interaction of α4β1 integrin with VCAM-1, as compared to natalizumab.

### MadCAM-1 Competitive Binding by ELISA.

The aim of this assay is to test the ability of natalizumab to inhibit interaction of α437 integrin with its cognate receptor—MadCAM-1 protein in a dose-dependent manner.

Constant amount of the coated α4β7 integrin is incubated with serial dilutions of natalizumab in the presence of Fc-tagged MadCAM-1 receptor. natalizumab and MadCAM-1 receptor now compete for binding to solid-phase associated α4β7 integrin. The higher the natalizumab concentration the more MadCAM-1 is inhibited from binding to α4β7 integrin. The lowest signal result is observed when no natalizumab is present. Bound natalizumab is subsequently detected with a POD-conjugated anti-human IgG antibody and a TMB-substrate reaction at the end of the assay.

Data are analyzed with 4PL fitting model. Final result is expressed as a Relative Potency (REP) of tested sample in relation to reference standard. The method variability was determined at the level of 8% coefficient variation (CV) of intermediate precision within the qualification exercise. Additionally accuracy, linearity and specificity were tested.

TABLE 6
MadCAM-1-integrin binding results by competitive ELISA

| | MadCAM-1 competitive ELISA | | | | | |
|---|---|---|---|---|---|---|
| | rep. | Average | | | | |
| Sample | No (n) | Results | min | max | SD | CV |
| Clone 1 | 3 | 0.810 | 0.738 | 0.857 | 0.063 | 7.82 |
| Clone 2 | 3 | 0.831 | 0.79 | 0.858 | 0.036 | 4.37 |
| Referent | 3 | 0.998 | 0.922 | 1.083 | 0.051 | 5.12 |
| 9 batches | | | | | | |

### Example 6 N-Glycosylation Profile Analysis with RapiFluor-MS Labeling Using Ultra Performance Liquid Chromatography with Fluorescence Detection

It was decided to reproduce Example 4 using a different fluorescence labeling, the RapiFluor-MS reagent, followed by high-performance hydrophilic interaction liquid chromatography with fluorescence detection (HILIC).

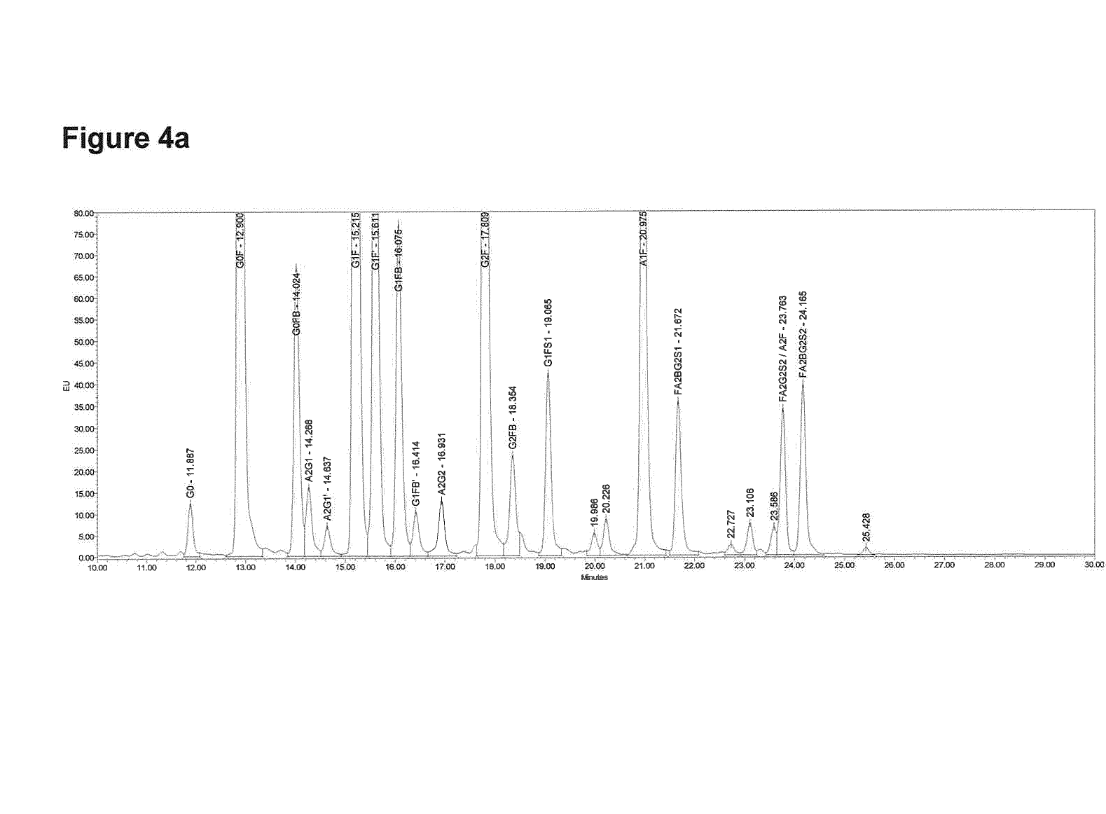

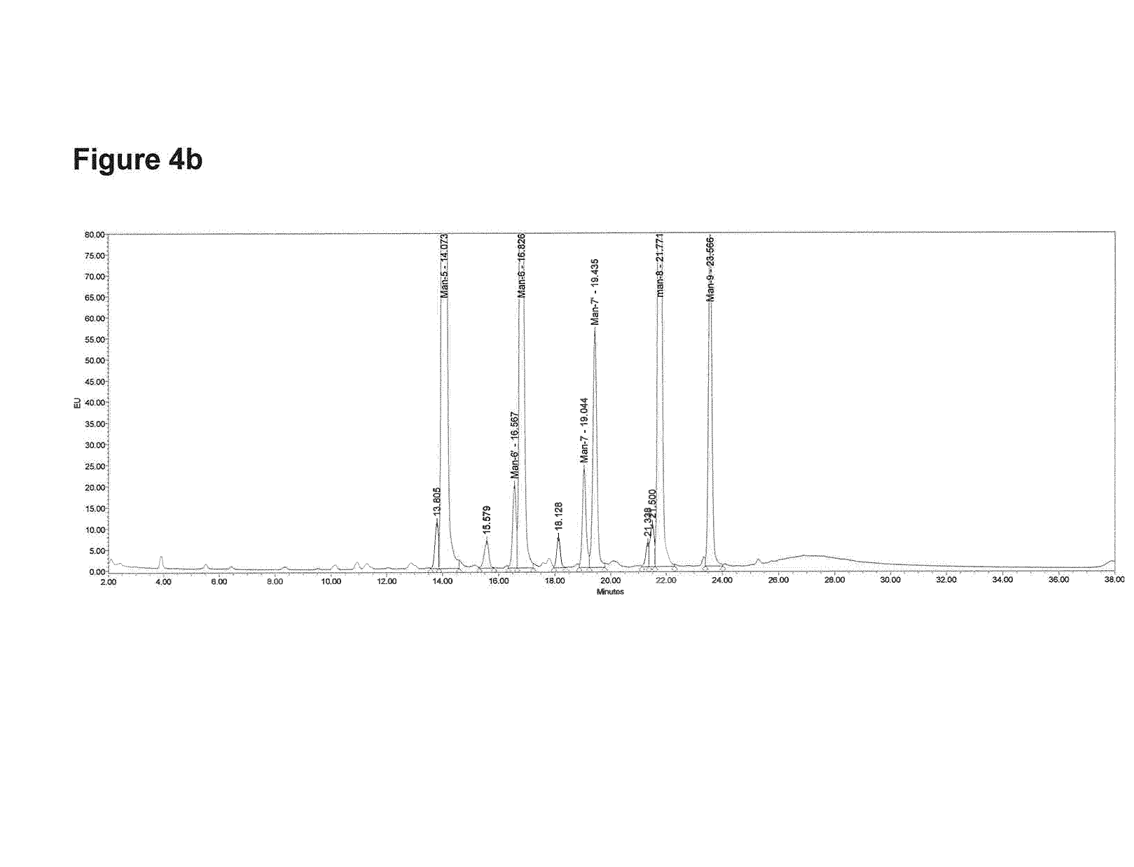

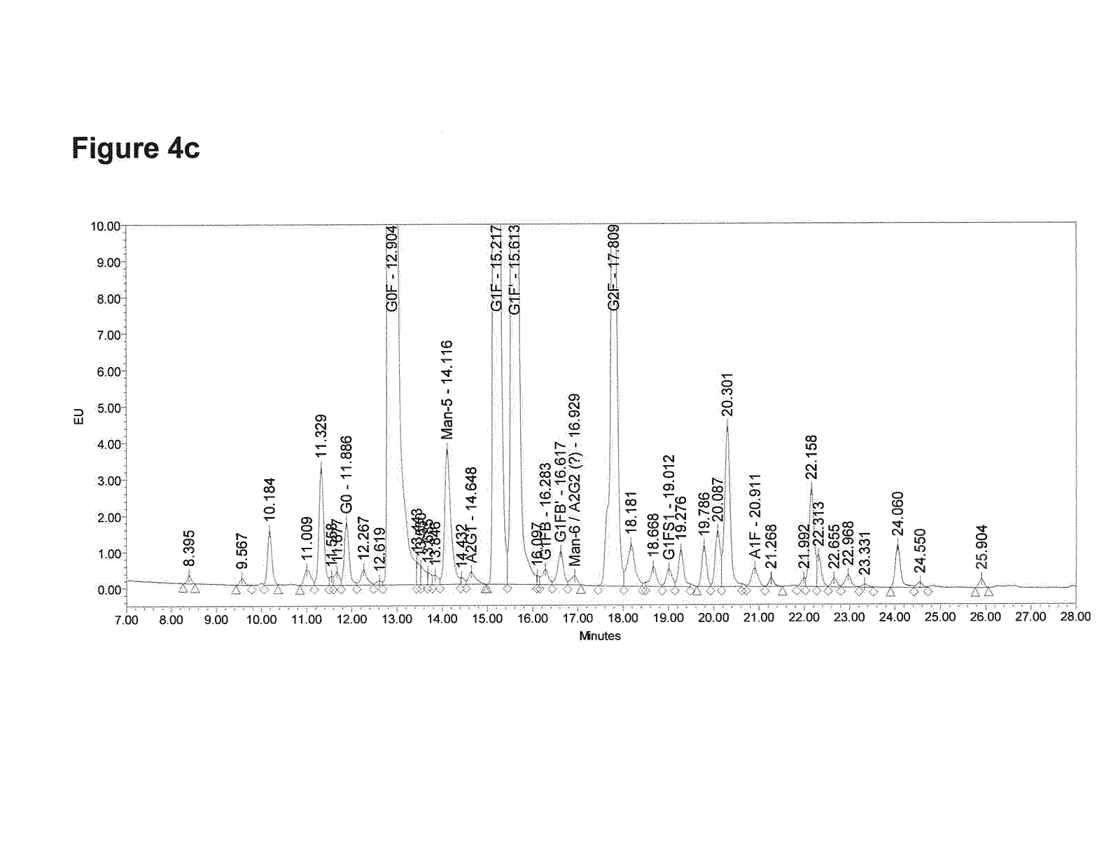

The above described approach let us identify 22 of N-linked glycan's, of which the 14 major peaks are shown in Table 7b. The identification of glycans is performed by the order of elution and GU value of peaks from RapiFluor-MS Glycan Performance Test Standard solution (GPTS) and Ribonuclease B solution (FIGS. 4a and 4b). The names and structure of identified glycans are presented in Table 7a. Sample preparation protocol for release of N-glycans consists of four steps:
1) Deglycosylation—in this step we use high temperature and RapiGest for denaturation and Rapid PGNase F for deglycosylation. Glycans are released from glycoprotein as glycosylamines. Briefly, 7.5 μl of 2 mg/ml solution of glycoprotein in water are provided in 1 ml tubes in duplicates or triplicates. Add 15.3 μl of ultrapure water as well as 6 μl of buffered solution containing 5% (w/v) RapiGest SF (RapiGest in GlycoWorks Rapid Buffer, GlycoWorks RapiFluor-MS N-Glycan Starter Kit P/N 176003635/GlycoWorks RapiFluor-MS N-Glycan Kit P/N 176003606). This mixture is then heat-denatured for 3 minutes in a heatblock at 90° C. After cooling to room temperature 1.2 μl Rapid PNGaseF is added, and incubated for 5 minutes at 50° C.
2) Labeling of glycosylamines—In this step the mixture of RapiFluor-MS Reagent and anhydrous DMF is used. RapiFluor-MS Reagent is a highly reactive primary/secondary amine labeling reagent. It hydrolyzes in water with a half-life on the order of 10-100 secs. It is therefore important that the reagent be dissolved in the anhydrous DMF, a non-nucleophilic, polar aprotic solvent. For successfully extending the labeling reaction samples need to be incubate with labeling mixture at least 5 min. In particular, the reagent solution is prepared by dissolving one vial of RapiFluor-MS in anhydrous DMF according to the manufacturer's protocol. Then, 12 μl of the RapiFluor-MS reagent is added to the deglycosylation mixture, and the reaction is allowed to proceed for 5 minutes at room temperature. Following incubation, the reaction is diluted with 358 μl of acetonitrile in preparation of the next step.
3) HILIC SPE clean-up of labeled glycosylamines—for clean-up of labeled glycosamines the GlycoWorks HILIC μElution Plate with vacuum manifold system is used. The plates are first equilibrated with 200 μl ultrapure water followed by 200 μl of water:acetonitrile 15:85 (v/v) for in total three times. Following loading of the complete samples from step 2, the samples are cleaned two times with 600 μl of a mixture of formic acid/water/acetonitrile 1:9:90 (v/v/v). The glycans are then eluted with 200 mM ammonium acetate in 5% acetonitrile. This elution buffer has been developed to deliver optimal, unbiased recovery of both small neutral glycans as well as high molecular weight, heavily sialylated glycans.
4) Preparing labeled glycans for HILIC-FLR analysis—in this step 90 μl of the eluate of glycans is, diluted with 100 μl of DMF and 210 μl of acetonitrile prior to HILIC chromatography.

The labeled RapiFluor-MS glycan composition was separated and determined by HILIC-UPLC measurement. The chromatographic separation was carried out on UPLC H-Class Bio System using column containing Waters amide bonded, Ethylene bridged Hybrid Technology (BEH) particles and fluorescent detection (excitation at 265 nm and emission at 425 nm) under Empower™ Software control. The Waters BEH Glycan (1.7 μm, 4.6 mm i.d.×150 mm) was used for testing applying eluents: A: Acetonitrile, and B: 50 mM Ammonium formate solution with pH 4.4 (prepared from concentrate). The glycans were separated using a linear gradient from 25% B to 46% B in 35 min with a flow rate of 0.4 ml/min, and the column temperature was 60° C. Gradient was followed by a washing step of 100% eluent B in 3 min and re-equilibration with 75% solvent A. The total run time was 55 min. Waters Empower 3 software with AppexTrack algorithm and GPC Technique was used for data evaluation. The peak assignment was performed following calibration of retention times to GU values. The sample composition was determined by detecting peaks based on their GU value and the relative proportions of each peak were calculated from the peak areas. Exemplary chromatograms of standard solutions are presented in FIGS. 4a and 4b. Exemplary chromatogram of test solution (reference product batch) is presented on FIG. 4c. Exemplary results of N-glycan content for reference product (range based on 9 batches testing) and tested product from clone selection step are presented in Table 7b.

The data of this experiment independently confirms the results shown in Example 4 with regard to glycans G0, G0F, G1F, G1F′, Man-5, and G2F.

Profiling of total glycans which are cleaved from the glycoprotein is the most common approach for characterizing protein glycosylation and allows to obtain Information about the various populations of glycans which are present on a glycoprotein (cf. General Monography of US Pharmacopeia USP38 of May 1, 2016, p. 1171). Depending on the chosen analytical method, prior derivatization/labeling may be needed to allow for the detection of certain glycans, including sialyl residues. Many protocols are available, and most of the steps in the analysis are well established. Because of the variety of available analytical techniques, a direct comparison of results obtained by different platforms is not always possible. Thus, the skilled person will not see the fact that glycans G0FB, G1FB, Man-6, G2, G2FB, G1FS1, and AF1 could not be detected using this alternative method as contradicting the results of Example 4.

### LIST OF REFERENCES

EP 2 202 307 A1
WO 2013/006461
WO 2009/009523
WO 95/19790
G. Urlaub, E. Kas, A. M. Carothers, and L. A. Chasin, “Deletion of the Diploid Dihydrofolate Locus from Cultured Mammalian Cells.” Cell, 33: 405-412 (1983).
G. Urlaub, P. J. Mitchell, E. Kas, L. A. Chasin, V. L. Funanage, T. T. Myoda and J. L. Hamlin, “The Effect of Gamma Rays at the Dihydrofolate Reductase Locus: Deletions and Inversions,” Somatic Cell and Molec. Genet., 12: 555-566 (1986).
W. Zhou, C.-C. Chen, B. Buckland and J. Aunins, “Fed-Batch Culture of Recombinant NS0 Myeloma Cells with High Monoclonal Antibody Production,” Biotechnology and Bioengineering, 55(5): 783-792 (1997).

TABLE 7b
N-glycan content results

| | rep. | | | | | | | | | | | | | | | |
|---|---|---|---|---|---|---|---|---|---|---|---|---|---|---|---|---|
| | No | Gly- | | | | | | | | | | | | | | |
| Sample | (n) | cans | G0 | G0F | G1 | G1F | G1F′ | G1FB | G1FB′ | Man-5 | Man-6 | Man-7 | G2F | G2FB | G1FS1 | A1F |
| Clone 1 | 3 | AV | 0.59 | 50.54 | 0.14 | 18.02 | 18.75 | not | not | 0.70 | not | not | 7.86 | not | not | not |
| | | | | | | | | detected | detected | | detected | detected | | detected | detected | detected |
| | | min | 0.58 | 50.17 | 0.14 | 17.98 | 18.69 | not | not | 0.67 | not | not | 7.75 | not | not | not |
| | | | | | | | | detected | detected | | detected | detected | | detected | detected | detected |
| | | max | 0.60 | 51.06 | 0.14 | 18.04 | 18.85 | not | not | 0.73 | not | not | 7.94 | not | not | not |
| | | | | | | | | detected | detected | | detected | detected | | detected | detected | detected |
| | | sd | 0.010 | 0 .465 | 0.000 | 0.035 | 0.085 | not | not | 0.031 | not | not | 0.119 | not | not | not |
| | | | | | | | | detected | detected | | detected | detected | | detected | detected | detected |
| | | CV | 1.69 | 0.92 | 0.00 | 0.19 | 0.45 | not | not | 4.39 | not | not | 1.52 | not | not | not |
| | | | | | | | | detected | detected | | detected | detected | | detected | detected | detected |
| Referent | 3 | AV | 0.31 | 53.32 | 0.13 | 14.90 | 15.21 | 0.25 | 0.38 | 1.18 | 0.12 | 0.21 | 6.33 | 0.17 | 0.23 | 0.13 |
| (9 | | min | 0.20 | 47.25 | 0.12 | 12.26 | 12.26 | 0.22 | 0.31 | 1.03 | 0.10 | 0.20 | 3 99 | 0 17 | 0.20 | 0.12 |
| batches) | | max | 0.53 | 63.62 | 0.13 | 16.29 | 16.92 | 0.27 | 0.45 | 1.38 | 0.15 | 0.23 | 7.93 | 0.17 | 0.28 | 0.13 |
| | | sd | 0.120 | 6.348 | 0.002 | 1.517 | 1.785 | 0.021 | 0.058 | 0.134 | 0.018 | 0.013 | 1.590 | 0.000 | 0.032 | 0.007 |
| | | RSD | 0.38 | 0.12 | 0.02 | 0.10 | 0.12 | 0.09 | 0.15 | 0.11 | 0.15 | 0.06 | 0.25 | 0.00 | 0.13 | 0 06 |

1. A cell culture obtainable from CHO DG44 cells which are capable of being cultured under serum-free or protein-free culture conditions, and which express a polypeptide comprising amino acids 19 to 231 of SEQ ID NO: 2 and a polypeptide comprising amino acids 19 to 468 of SEQ ID NO: 4.

2. The cell culture of claim 1, wherein the cells express a polypeptide comprising the amino acid sequence of SEQ ID NO: 2 and a polypeptide comprising the amino acid sequence of SEQ ID NO: 4.

3. The cell culture of claim 1, wherein said expressed polypeptides have a N-glycan content comprising:
(i) 36-61% of the asialo-, agalacto-biantennary type; and
(ii) 25.5-36.5% of the asialo-, mono-galactosylated-biantennary type and has a core substituted with fucose; and
(iii) 5-11.5% of the asialo-, galactosylated biantennary type; and
(iv) 0.8-3.5% of the oligomannose 5 and oligomannose 6 type;
as determined using high-performance hydrophilic interaction liquid chromatography with fluorescence detection (HILIC).

4. The cell culture of claim 3, wherein 36.5-60% of the asialo-, agalactosylated-biantennary type has a core substituted with fucose.

5. The cell culture of claim 4, wherein 0.3-0.9% of the asialo-, agalactosylated-biantennary type has a core substituted with fucose and has a bisecting N-acetylglucosamin.

6. The cell culture of claim 3, wherein 0.05-0.48% of the asialo-, mono-galactosylated-biantennary type which has a core substituted with fucose has a bisecting N-acetylglucosamin.

7. The cell culture of claim 3, wherein 4.9-11% of the asialo-, galactosylated-biantennary type has a core substituted with fucose.

8. The cell culture of claim 7, wherein 0.05-0.5% of the asialo-, galactosylated-biantennary type has a core substituted with fucose and has a bisecting N-acetylglucosamin.

9. The cell culture of claim 3, wherein said expressed polypeptides have a N-glycan content comprising:
0.5-3.1% of the oligomannose 5 type;
0.1-0.35% of the oligomannose 6 type; or
0.5-3.1% of the oligomannose 5 type and 0.1-0.35% of the oligomannose 6 type.

10. The cell culture of claim 1, wherein said expressed polypeptides have a N-glycan content comprising:
(i) 36.5-60% of the asialo-, agalactosylated-biantennary type and having a core substituted with fucose and
(ii) 24.5-37.5% of the asialo-, mono-galactosylated-biantennary type and having a core substituted with fucose and without a bisecting N-acetylglucosamin; and
(iii) 3.5-10.5% of the asialo-, galactosylated-biantennary type having a core substituted with fucose and without a bisecting N-acetylglucosamin; and
(iv) 0.5-3.1% of the oligomannose 5 type;
as determined using high-performance hydrophilic interaction liquid chromatography with fluorescence detection (HILIC).

11. A cell of the cell culture according to claim 1.

12. A method for producing a therapeutic monoclonal antibody, comprising the steps of:
(a) cultivating a cell culture according to claim 1; and
(b) recovering the polypeptide comprising amino acids 19 to 231 of SEQ ID NO: 2 and the polypeptide comprising amino acids 19 to 468 of SEQ ID NO: 4 from said cell culture.

13. The method of claim 12, wherein the therapeutic antibody is natalizumab.

14. (canceled)

15. (canceled)

16. The cell culture of claim 3, wherein said expressed polypeptides have a N-glycan content comprising:
(i) 47-57% of the asialo-, agalacto-biantennary type; and
(ii) 30-35% of the asialo-, mono-galactosylated-biantennary type and has a core substituted with fucose; and
(iii) 5.1-10% of the asialo-, galactosylated biantennary type; and
(iv) 1.0-3.2% of the oligomannose 5 and oligomannose 6 type;
as determined using high-performance hydrophilic interaction liquid chromatography with fluorescence detection (HILIC).

17. The cell culture of claim 4, wherein 40-58% of the asialo-, agalactosylated-biantennary type has a core substituted with fucose.

18. The cell culture of claim 5, wherein 0.35-0.85% of the asialo-, agalactosylated-biantennary type has a core substituted with fucose and has a bisecting N-acetylglucosamin.

19. The cell culture of claim 6, wherein 0.1-0.47% of the asialo-, mono-galactosylated-biantennary type which has a core substituted with fucose has a bisecting N-acetylglucosamin.

20. The cell culture of claim 7, wherein 5-9% of the asialo-, galactosylated-biantennary type has a core substituted with fucose.

21. The cell culture of claim 8, wherein 0.1-0.45% of the asialo-, galactosylated-biantennary type has a core substituted with fucose and has a bisecting N-acetylglucosamin.

22. The cell culture of claim 9, wherein said expressed polypeptides have a N-glycan content comprising:
0.6-2.9% of the oligomannose 5 type; or
0.11-0.3% of the oligomannose 6 type; or
0.6-2.9% of the oligomannose 5 type and 0.11-0.3% of the oligomannose 6 type.


SEQUENCE LISTING (4 sequences)

SEQ ID NO: 1 - DNA, 699 bp, Artificial
  Light Chain Natalizumab
  sig_peptide (1)..(54): SP(3)
atgaagtgggtgaccttcatctccctgctgtttctgttctcctccgcctactccgacatccagatgacccagtccccctccagcctgtccgcctccgtgggcgacagagtgaccatcacatgcaagacctcccaggacatcaacaagtacatggcctggtatcagcagacccccggcaaggcccctcggctgctgatccactacacctccgccctgcagcccggcatcccttccagattctccggctctggctctggccgggactacaccttcaccatctccagcctgcagcctgaggacattgccacctactactgcctgcagtacgacaacctgtggaccttcggccagggcaccaaggtggaaatcaagcggaccgtggccgctccctccgtgttcatcttcccaccctccgacgagcagctgaagtccggcaccgccagcgtggtgtgcctgctgaacaacttctacccccgcgaggccaaggtgcagtggaaggtggacaacgccctgcagagcggcaactcccaggaatccgtgaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgctgatag

SEQ ID NO: 2 - protein, 231 aa, Artificial
  Light Chain Natalizumab
  SIGNAL (1)..(18): SP(3)
  mat_peptide (19)..(231)
MKWVTFISLLFLFSSAYSDIQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1410 bp, Artificial
  Heavy Chain Natalizumab
  sig_peptide (1)..(54): SP(3)
atgaagtgggtgaccttcatctccctgctgtttctgttctccagcgcctactcccaggtgcagctggtgcagtctggcgccgaagtgaagaaacctggcgcctccgtgaaggtgtcctgcaaggcctccggcttcaacatcaaggacacctacatccactgggtgcgacaggcccctggccagcggctggaatggatgggcagaatcgaccccgccaacggctacactaagtacgaccccaagttccagggcagagtgaccatcaccgccgacacctccgcctccaccgcctacatggaactgtcctccctgcggagcgaggacaccgccgtgtactactgcgccagagagggctactacggcaactacggcgtgtacgccatggactactggggccagggcaccctggtgacagtgtcctccgccagcaccaagggcccctccgtgttccctctggccccttgctcccggtccacctccgagtctaccgccgctctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccatccagctccctgggcaccaagacctacacctgtaacgtggaccacaagccctccaacaccaaggtggacaagcgggtggaatctaagtacggccctccctgccccagctgccctgcccctgaattcctgggcggaccttccgtgttcctgttccccccaaagcccaaggacaccctgatgatctcccggacccccgaagtgacctgcgtggtggtggacgtgtcccaggaagatcccgaggtgcagttcaattggtacgtggacggcgtggaagtgcacaacgccaagaccaagcccagagaggaacagttcaactccacctaccgggtggtgtctgtgctgaccgtgctgcaccaggactggctgaacggcaaagagtacaagtgcaaggtgtccaacaagggcctgcccagctccatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgaccgtggacaagtcccggtggcaggaaggcaacgtgttctcctgctccgtgatgcacgaggccctgcacaaccactacacccagaagtccctgtccctgagcctgggcaagtgatag

SEQ ID NO: 4 - protein, 468 aa, Artificial
  Heavy Chain Natalizumab
  SIGNAL (1)..(18): SP(3)
  mat_peptide (19)..(468)
MKWVTFISLLFLFSSAYSQVQLVQSGAEVKKPGASVKVSCKASGFNIKDTYIHWVRQAPGQRLEWMGRIDPANGYTKYDPKFQGRVTITADTSASTAYMELSSLRSEDTAVYYCAREGYYGNYGVYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK